(19) United States
(12) Patent Application Publication
(10) Pub. No.: US 2025/0043251 A1
(43) Pub. Date: Feb. 6, 2025

(54) METHOD FOR FURTHER UPSCALING THE LARGE-SCALE PRODUCTION OF THE ONCOLYTIC H-1 PROTOPARVOVIRUS (H-1PV) USING A CARRIER-BASED PRODUCTION PROCESS COMBINED WITH AN OPTIMIZED CELL CULTURE MEDIUM

(71) Applicant: DEUTSCHES KREBSFORSCHUNGSZENTRUM STIFTUNG DES ÖFFENTLICHEN RECHTS, Heidelberg (DE)

(72) Inventors: Barbara LEUCHS, Heidelberg (DE); Veronika FREHTMAN, Heidelberg (DE); Martin VOGEL, Heidelberg (DE); Daniel WOHLFARTH, Heildelberg (DE); Linh Minh Phuc PHAN, Heidelberg (DE); Adrian BRUNECKER, Heidelberg (DE)

(21) Appl. No.: 18/610,482

(22) Filed: Mar. 20, 2024

Related U.S. Application Data
(63) Continuation-in-part of application No. PCT/EP2022/076992, filed on Sep. 28, 2022.

(30) Foreign Application Priority Data
Sep. 30, 2021 (EP) 21200181.2

(51) Int. Cl. C12N 7/00; B01D 15/36; B01D 61/14; C12N 5/071

(52) U.S. Cl. CPC C12N 7/00; B01D 15/363; B01D 61/146; C12N 5/0686; C12N 2500/32; C12N 2500/34; C12N 2500/92; C12N 2750/14351

(57) ABSTRACT

The present invention provides a method for upstream optimization the large-scale parvovirus production, preferably the oncolytic protoparvovirus H-1 (H-1PV). It is based on microcarriers or macrocarriers and their respective use in suspension or fixed-bed, an optimized cell culture medium, and a medium exchange strategy. In summary, with the optimized cell culture medium and the new medium exchange strategy, the inventors established a reduction in seeded cell density and animal serum, leading to an animal serum-free harvest. The tested carriers are best suited for a high H-1PV yield, cell growth, and bead-to-bead transfer capability, wherein the inventors additionally scaled up the process from 24-well plates to Erlenmeyer, Spinner flask and iCellis nano. As a conclusion, the present invention provides a large-scale method for producing the oncolytic protoparvovirus H-1 with a high virus yield, while lowering production costs and avoiding undesired products of animal origin at the same time.

### RELATED APPLICATIONS AND INCORPORATION BY REFERENCE

This application is a continuation-in-part application of international patent application Serial No. PCT/EP2022/076992 filed Sep. 28, 2022, which published as PCT Publication No. WO 2023/052429 on Apr. 6, 2023, which claims benefit of European patent application Serial No. 21200181.2 filed Sep. 30, 2021.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention provides a method for the upstream optimization of the large-scale parvovirus production, preferably the oncolytic protoparvovirus H-1 (H-1PV). It is based on microcarriers or macrocarriers and their respective use in suspension or fixed-bed, an optimized cell culture medium, and a new medium exchange strategy. In summary, with the optimized cell culture medium and the new medium exchange strategy, the inventors established a reduction in seeded cell density and animal serum, leading to an animal serum-free harvest. The tested carriers are best suited for a high H-1PV yield, cell growth, and bead-to-bead transfer capability, wherein the inventors additionally tested production in Erlenmeyer, Spinner flasks and iCellis nano. As a conclusion, the present invention provides a large-scale method for producing the oncolytic protoparvovirus H-1 with a high virus yield, while lowering production costs and avoiding undesired products of animal origin at the same time.

### BACKGROUND OF THE INVENTION

According to the World's Health Organization (WHO), cancer was the second leading cause of death worldwide in 2018, with an estimated 9.6 million deaths. The economic damage associated with cancer is significant and increasing, totaling approximately $1.16 trillion in 2010 alone (Stewart 2014). Oncolytic virus (OV) therapy represents a promising approach for treating this disease. OVs are genetically engineered or naturally occurring viruses that selectively destroy cancer cells without harming healthy tissue (Fukuhara 2016). In 2015, the first OV therapeutic (T-VEC or Imlygic™) was approved by the FDA and followed sometimes later by the EMA. Other OV therapeutics based on different virus platforms (webs.iiitd.edu.in/raghava/ovirustdb/clinical.php) are in the development pipeline.

The OV drug “ParvOryx®” utilizes the wild-type parvovirus H-1PV, which belongs to the genus Protoparvovirus within the Parvovirinae subfamily of Parvoviridae (Cotmore 2014). It demonstrated oncolytic and oncosuppressive properties during preclinical proof-of-concept studies in various cultured cell lines, in animal (Rommelaere 2010; Nuesch 2012) and xenograft models against several human tumor species (Geletneky 2010; Faisst 1998; Angelova 2009b; Dupressoir 1989; Angelova 2009a). H-1PV also showed safety and immunogenic activity in clinical phase I/IIa studies (Geletneky 2012; Geletneky 2017) and phase II studies (Hajda 2021).

Thus, patient treatment with the oncolytic protoparvovirus H-1PV has shown promising results in clinical trials against several types of cancers (Hajda 2021; Geletneky 2017). In preparation for market release of H-1PV in the future, however, the culture medium and production process has to be adapted and optimized to a robust and controlled large-scale, i.e. according to system parameters such as agitation speed, aeration, seeding cell density, time of infection, multiplicity of infection, time of harvest and harvest strategy.

Therefore, see for example, the European patent No. 3313987 that relates to a method of producing parvovirus particles using the producer cell line NB-324K, wherein the method uses animal component-containing cell culture medium for large-scale virus production. In order to eliminate unwanted contaminants, such as animal-derived components, an improved infectious particle purification has been introduced, which is based on two subsequent iodixanol density gradient ultracentrifugation steps, the first in PBS and the second in Ringer. Furthermore, it is described in the European patent application EP 17771376.5 that a further optimized process for parvovirus production mainly based on the animal component-free cell culture medium VP-SFM™allows easier and more cost-effective production and purification of the parvovirus produced by NB-324K cells.

However, none of the approaches provided a large-scale method for producing the oncolytic protoparvovirus H-1 with a high virus yield, while lowering production costs and avoiding undesired products of animal origin at the same time.

Thus, the technical problem underlying the present invention is to further optimize parvovirus large-scale production.

The solution is achieved by providing the embodiments characterized in the claims.

### SUMMARY OF THE INVENTION

Patient treatment with the oncolytic protoparvovirus H-1PV has shown promising results in clinical trials against several types of cancers (Hajda 2021; Geletneky 2017). These data show the first results for producing H-1PV in a large-scale bioreactor system. The process, however, needs to be adapted and optimized according to system parameters, such as agitation speed, aeration, seeding cell density, time of infection, multiplicity of infection, and time of harvest.

Initially, the previously described cell culture medium requires the supplementation with animal serum as being essential for cell growth, metabolism, and to stimulate proliferation. Thus, the addition of animal serum, such as fetal bovine serum (FBS) in cell culture is still common, but problematic in terms of quality, lot-to-lot reproducibility, animal welfare, supply, cost (van der Valk 2018), and potential regulatory restrictions in the future. Therefore, the method of the present invention uses the optimized culture medium VP-SFM™, suitable for FBS-free parvovirus production (Liu 2017; Rourou 2007; Martinez 2020) and an at least two-step medium exchange strategy in order to reduce the required amount of FBS by up to 80% for a FBS-free harvest at a comparable virus yield. With an additional medium exchange and a three-step FBS reduction from e.g., 2% to 1% and to 0%, a production yield boost of approximately 0.3 log was achieved comparing to FBS reduction from 2% to 0% and thereafter a second time to 0%, while still reducing the FBS needed by up to 40%. By applying the at least two-step medium exchange strategy, preferably by applying the three-step medium exchange strategy, during the experiments resulting in the present invention, a high virus production yield with a FBS-free harvest and fewer impurities for the downstream process could be achieved.

Furthermore, using the method according to the present invention, more specifically the non-simultaneous seeding and infection process, the seeding density could be reduced from 7.9E3 cells/cm² to 5.0E3 cells/cm² while a similar virus yield after adapting to VP-SFM™ has been maintained. In this way, the cell production costs could be lowered because the expansion time could be reduced and fewer resources were needed for the seed train.

Moreover, in the prior art it is common to employ microcarriers for suspension culture or macrocarriers for a fixed-bed bioreactor for large-scale H-1PV production with adherent cells, such as with the producer cell line NB-324K. In large-scale production methods, the implementation of freeze-thaw cycles employed in the past is difficult so that the method of the present invention is performed without the need for a trypsination step and freeze-thaw cycles. Instead, the inventors formerly introduced a Triton® X-100 (key ingredient: 2-[4-(2,4,4-trimethylpentan-2-yl)phenoxy]ethanol)-based chemical cell lysis. With Triton® X-100 lysis, satisfactory production yields have been achieved on different microcarriers and macrocarriers. Currently, however, the detergent Triton® X-100 is considered eco-toxic by regulatory authorities (https://echa.europa.eu/authorisation-list). Thus, in one embodiment of the present invention, lysis buffer containing Tris, MgCl₂ and recombinant cell-dissociation enzyme TrypLE™ with Tween® 80 has been tested as an alternative lysis buffer because it is eco-friendlier than Triton® X-100 buffer, wherein also Tween® 80 may be omitted from the lysis buffer recipe without negative influence on the virus yield.

During the experiments resulting in the present invention, several microcarriers and macrocarriers have been tested. Initially, in one embodiment of the present invention, the solid microcarrier Cytodex® 1, which is a cross-linked dextran matrix with positively charged DEAE (N,N-diethylaminoethyl)-groups distributed throughout the matrix, has been found to be well suited in large-scale productions or enhanced attachment CellBIND® (EA) microcarrier. The enhanced attachment CellBind® (EA) microcarrier has a negative surface charge due to oxygen-containing functional groups incorporated in the polystyrene surface. These microcarriers showed a promising bead-to-bead cell transfer capability during cell growth and virus production. To date, Cytodex® 1 has been reported for adenoviruses and retroviruses (Wu 2002), vaccinia virus (Liu 2017), and influenza virus A (Tree 2001).

An alternative embodiment of the method of the present invention is represented by a polypropylene and polyester nonwoven fiber (Fibra-Cel®) or a nonwoven, hydrophilized polyethylene terephthalate (PET) macrocarrier (iCELLis®) because their use resulted in a good production yield in suspension cultures, wherein these macrocarriers are also designed for fixed-bed bioreactors, in which a higher yield may be achieved.

In summary, in the experiments resulting in the present invention, the inventors optimized the cell culture medium and applied a new medium exchange strategy. Finally, they established a reduction in seeded cell density and supplementation of animal serum, leading to an animal component-free harvest. Thus, the cell production costs could have been lowered because the expansion time and the amount of impurities could be reduced and fewer resources were needed for the seed train. Furthermore, they tested the microcarriers and macrocarriers best suited for a high H-1PV yield, cell growth, and bead-to-bead transfer capability and successfully scaled up the process from 24-well plates to Erlenmeyer and Spinner flasks, wherein production in large stirred tanks bioreactors with microcarrier or fixed bed bioreactors e.g., the iCELLis® 500 m², seems to be promising.

In summary, the present invention relates to a method for producing parvovirus H-1, said method comprising:
(a) providing the producer cell line NB-324K;
(b) producing the seed train by growing the NB-324K cells under suitable conditions;
(c) providing a culture vessel containing animal component-free cell culture medium supplemented with 2% animal serum and a microcarrier or a macrocarrier at the seeding time point;
(d) seeding the NB-324K cells of step (b) in the culture vessel of step (c);
(e) cell expansion of the NB-324K cells;
(f) 100% medium exchange at the infection time point with animal component-free cell culture medium including microcarrier or macrocarrier, wherein the animal component-free cell culture medium is supplemented with 1% animal serum or is without animal serum;
(g) harvesting the NB-324K cells 3 to 8 days post-infection with lysis buffer containing 1-100 mM Tris, 1-10 mM MgCl₂, 2.5-10% TrypLE™, pH 9-10 with or without 0.1-1% Tween® 80, and agitation of the lysis buffer, followed by wash with wash buffer containing 1-100 mM Tris, 1-10 mM MgCl₂, pH 9-10, wherein the lysis and wash buffers are pooled together;
(h) clarifying the parvovirus harvest by filtration;
(i) eliminating non-encapsidated viral DNA and contaminating host cell DNA by DNAse treatment;
(j) tangential flow filtration for buffer exchange and concentration;
(k) anion exchange chromatography to eliminate empty particles and most impurities;
(l) tangential flow filtration for buffer exchange to 0-3% Visipaque and 97-100% Ringer solution and concentration;
(m) final formulation in 48% Visipaque/Ringer solution.

In a preferred embodiment, in step (g) the lysis buffer contains 25 mM Tris, 5 mM MgCl₂, 5% TrypLE™, pH 10.

In another preferred embodiment, in step (g) the step of washing is performed with buffer containing 25 mM Tris, 5 mM MgCl₂ pH 10 and, wherein step (g) results in buffer containing 25 mM Tris, 5 mM MgCl₂, 2.5% TrypLE™, with or without 0.1-1% Tween® 80, preferably less than 0.25% Tween® 80.

In another preferred embodiment, in step (g) the treatment with lysis buffer is performed for 1 h at 40° C. without (w/o) CO₂ shaken with 70 rpm or circulated in bioreactor and treatment with wash buffer is performed for 1 min w/o CO₂ shaken with 70 rpm or circulated in bioreactor.

### BRIEF DESCRIPTION OF THE DRAWINGS

FIG. 1: Overview of process time lines and FBS content using VP-SFM™ medium.

FIG. 2: Similar H-1PV-specific virus yield with 5% FBS or two-step FBS reduction scheme. Here, 3.6E4 NB-324K cells/cm² were seeded in VP-SFM™ medium supplemented with 5% or 2% FBS. After 3 days of cell expansion, the infection with a MOI of 0.05 and a 100% medium exchange with 5% or 0% FBS was performed. Cells were harvested and lysed 4 days postinfection.

FIG. 3: Boost of H-1PV-specific virus yield with three-step FBS reduction using VP-SFM™. Here, 5E3 NB-324K cells/cm² were seeded with 2% FBS. After 3 days of cell expansion, the first 100% medium exchange to either 1% or 0% FBS and infection with a MOI of 0.01 was performed for production phase I from day 3-day 5. Two days postinfection a second 100% medium exchange was performed without FBS for production phase II: day 5-day 7. Cells were harvested and lysed 4 days postinfection on day 7.

FIG. 4: Comparison of harvest with Triton® X-100 process, the freeze-thaw process in Virus Tris (VT) buffer and 25 mM Tris, 5 mM MgCl₂, with or without 0.25% Tween 80 and with or without 2.5% TrypLE pH 10. NB-324K cells were seeded in VP-SFM™ with 2% FBS and 5E3 cells/cm² in 175 cm² flasks for 3 days, then 100% medium exchange to VP-SFM™ with 0% FBS and infection with MOI of 0.05 was performed. 4 days post infection cells were rinsed with 1×PBS before lysis and harvested with different lysis methods.

FIG. 5: Microscopic observation of cell bridges between confluent and freshly added microcarriers. a: Enhanced attachment (10×); b: Cytodex® 1 (10×); Scale bar=200 μm

FIG. 6a: H-1PV-specific virus yield screening with different carriers. Here, 4E4 NB-324K cells/cm² were seeded with 10 cm²/ml (11.2 cm²/ml for iC) growth area of each carrier in 24-well plates and shaken at 100 rpm. Cells were harvested and lysed 4 days post infection.

FIG. 6b: H-1PV-specific virus yield with different microcarriers after bead-to-bead transfer 2E4 [▪] or 4E4 [ [symbol] ] NB-324K cells/cm² were seeded with 5 cm²/ml growth area of each microcarrier in 24-well plates and orbitally shaken at 100 rpm. After 3 days of cell expansion, cells were infected with a MOI of 0.01, while growth surface and medium volume was doubled by addition of fresh microcarriers in fresh medium, followed by a 1:2 split to return to start conditions of medium volume and microcarrier density. Cells were harvested and lysed 4 days post infection.

FIG. 7: H-1PV-specific virus yield after scale-up of best performing microcarriers and macrocarriers. Here, 2E4 NB-324K cells/cm² were seeded with 10 cm²/ml growth area (a: microcarrier EA or CD1; b: macrocarrier iC or FC) in a VP-SFM™ volume of 40 ml per 125 ml Erlenmeyer flask [▪] or 100 ml per Spinner flask [ [symbol] ]. For seeding, a cycle of 1 min at 40 rpm, followed by 30 min at 0 rpm was repeated four times to a total seeding time of 2 h. Then, agitation was set between 30-100 rpm for the Erlenmeyer flask or 40 rpm for the Spinner flask. After 3 days of cell expansion, cells were infected with a MOI of 0.01 and 50% of the medium exchanged with fresh medium. Cells were harvested and lysed 4 days post infection.

FIG. 8: H-1PV-specific virus yield in iCELLis® nano with different fixed-bed and respective Tflask run controls. In separate bioreactor runs, 5E3 cells/cm² (0.53 m²) or 9E3 cells/cm² (4 m²) were seeded in iCELLis® nano fixed bed [▪] and Tflask run control [ [symbol] ]. After 3 days of cell expansion with VP-SFM™ Cell expansion medium, the first 100% medium exchange to VP-SFM™ Infection medium and infection with a MOI of 0.01 was performed for production phase I from day 3-day 5. Two days post infection a second 100% medium exchange was performed with VP-SFM™ w/o FBS medium for the production phase II: day 5 to day 7. Cells were harvested and lysed 4 days post infection on day 7.

### DETAILED DESCRIPTION OF THE INVENTION

The technical problem underlying the present invention is to further optimize large-scale production of oncolytic protoparvovirus production, preferably H-1PV, because to date none of the approaches currently available provide a large-scale method for producing H-1PV with a high virus yield, while lowering production costs and avoiding undesired products of animal origin at the same time.

To increase the virus yield, while lowering production costs and avoiding undesired products of animal origin, in the experiments resulting in the present invention, the animal component-free cell culture medium VP-SFM™ supplemented with 5% FBS during seeding and infection was compared to VP-SFM™ supplemented with 2% FBS during seeding and 0% FBS during infection.

In this respect, the term “cell culture” means the maintenance of cells in an artificial, in vitro environment. The media of the present invention can be used to culture adherent mammalian cell, i.e. a cell which adhere to the culture vessel, preferably epithelial cells, such as human newborn kidney (NB-324K) cells transformed with simian virus 40 (Tattersall 1983), representing the “producer cell line”.

The term “cultivation” or “growing” means the maintenance of cells in vitro under conditions favouring growth, differentiation or continued viability, in an active or quiescent state, of the cells.

The phrase “cell culture medium” refers to a nutritive solution for cultivating cells.

The term “ingredient” refers to any compound, whether of chemical or biological origin, that can be used in cell culture media to maintain or promote the growth of proliferation of cells. The terms “component”, “nutrient” and ingredient” can be used interchangeably and are all meant to refer to such compounds. Typical ingredients that are used in cell culture media include glucose, glutamine, glutamate, growth factors, insulin and proteins.

The term “animal component-free” or “serum-free” medium is one which contains no animal serum. A serum-free medium is distinguished from low-serum and essentially serum-free media, both of which contain serum. More specifically, the term “serum” or rather “animal-serum” refers to e.g., heat inactivated fetal bovine serum (FBS; Biowest, France).

The process of the present invention uses an “animal component-free cell culture medium” comprising the ingredients glucose, glutamine, glutamate, proteins (e.g., growth factors, insulin, etc.), wherein the medium is capable of supporting the cultivation of epithelial cells in vitro, preferably NB-324K cells, for the production of H-1PV.

The medium of the present invention can be used to grow human epithelial cells, preferably NB-324K cells, to high density and/or to increase parvovirus production. In one preferred embodiment, the process of the present invention uses an animal component-free cell culture medium containing about 16-22 mM glucose, 3-5 mM glutamine, 0.1-0.6 mM glutamate, 0.5-1.0 mM lactate, less than 0.3 mM ammonium and 3-10 mg/ml proteins, wherein the medium is capable of supporting the cultivation of NB-324K cells for the production of H-1PV.

Preferably, the animal component-free cell culture medium contains 17-20 mM glucose, about 4 mM glutamine, about 0.15-0.5 mM glutamate, about 0.7 mM lactate, less than 0.2 mM ammonium and 4-8 mg/ml proteins e.g., supplements, epithelial growth factor and insulin.

In a particular preferred embodiment, the animal component-free cell culture medium contains 19.14 mM glucose, 4.25 mM glutamine, 0.174 mM glutamate, 0.669 mM lactate, less than 0.05 mM ammonium and about 5 mg/ml proteins. These proteins include EGF, insulin and proteineous supplements. This medium is called VP-SFM™ (Thermofisher, USA).

Thus, according to the present invention, the NB-324K cells are cultured in VP-SFM^{T}M Medium. In a more specific embodiment, the NB-324K cells are cultured at 37° C. in a 5% CO₂ atmosphere.

In a further embodiment, the cell culture media are supplemented with 100 U/ml penicillin, 100 μg/ml streptomycin, and 3-5 mM L-glutamine (Thermofisher, USA). Finally, VP-SFM™ medium with 4 mM glutamine supplementation is preferred because higher concentrations do not result in increased virus yield, therefore lowering the production costs (see Table 1).

TABLE 1
Overview of media and supplementations

| Basal | Medium | FBS | Gln |
|---|---|---|---|
| medium | description | [%] | [mM] |
| VP-SFM ™ | full | 5 | 4 |
| | Cell expansion | 2 | 4 |
| | Infection | 1 | 4 |
| | w/o FBS | 0 | 4 |

All media were supplemented with 100 U/ml penicillin and 100 μg/ml streptomycin

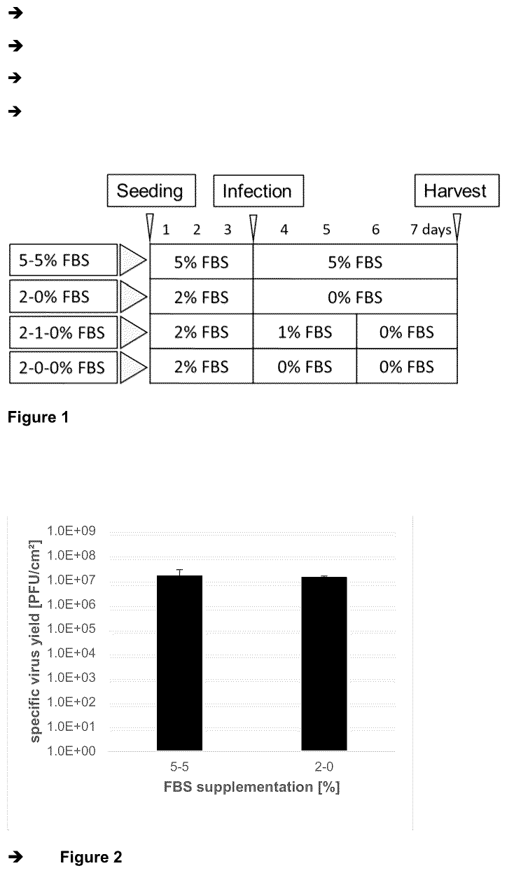

In the experiments resulting in the present invention, the NB-324K cells have been cultured in VP-SFM™ medium with 0%, 1%, 2%, or 5% FBS (see Table 1) but have been seeded with 5% or 2% FBS for production. Similar virus yields have been shown with VP-SFM™ supplemented with 5% FBS for seeding and production was compared to VP-SFM™ supplemented with 2% FBS for seeding and 0% FBS for production (see FIG. 2). Thus, according to the present invention, VP-SFM™ medium supplemented with 2% FBS is preferred, wherein the method of the present invention starts with 5% FBS in the seed train, preferably. As already stated above, VP-SFM™ medium with 2% FBS supplementation at seeding and 0% FBS in the infection is preferred compared to 5% FBS supplementation because it results in similar virus yield decreasing amounts of FBS to 80% (see FIG. 2). Thus, applying the presently described method for producing H-1PV with a high virus yield, the production costs can be lowered while undesired products of animal origin are avoided at the same time.

With regard to the virus infection in the method of the present invention, a virus stock of wild-type H-1PV is preferably used to infect the NB-324K cells, more preferably with a MOI of 0.5 to 5×10² plaque-forming units (PFU) per cell, or rather 0.01 or 0.05 PFU per cell (PFU/cell). According to the present invention, the NB-324K cells are infected with H-1PV at seeding (“simultaneous” seeding and infection) or after 2 to 6 days of cell expansion preferably at day 3 to 6 after the cell expansion of the NB-324K cells has been started (“non-simultaneous” seeding and infection).

In one specific embodiment of the present invention, the NB-324K cells are infected at a cell density from 2.0×10⁴ to 5.0×10⁴ cells/cm² with the parvovirus, wherein the time of infection (TOI) is either at seeding (“simultaneous” seeding and infection) or after 2 to 6 days of cell expansion, preferably at day 3 to 6 after the cell expansion of the NB-324K cells has been started (“non-simultaneous” seeding and infection).

For simultaneous seeding and infection, the NB-324K cells of the present invention are seeded in the culture vessel, in which animal component-free cell culture medium supplemented with 2% animal serum is laid before, wherein the animal component-free cell culture medium is VP-SFM™ medium. In one specific embodiment of the present invention, the NB-324K cells are infected at a cell density from 2.0×10⁴ to 5.0×10⁴ cells/cm², preferably 4.0×10⁴ cells/cm². In a further specific embodiment, the cells are harvested after 3 to 5 days post infection, preferably after 4 days post infection.

For non-simultaneous seeding and infection, the NB-324K cells of the present invention are seeded in the culture vessel, in which animal component-free cell culture medium supplemented with 2% animal serum is laid before, wherein the animal component-free cell culture medium is VP-SFM™ medium. In a specific embodiment, the inventors grew the cells then for 2 to 6 days, preferably 3 to 6 days (“cell expansion”). After 2 to 6 days, preferably 3 to 6 days of cell expansion, the animal component-free cell culture medium supplemented with 2% animal serum is completely exchanged (“100% medium exchange”) with new animal component-free cell culture medium supplemented with 0%, 1% or 2% FBS, preferably 0% or 1% FBS (“2-0-0% FBS” and “2-1-0% FBS” strategy), wherein the new animal component-free cell culture medium is VP-SFM™ medium. At the same time, the NB-324K cells are infected with a virus stock of wild-type H-1PV. In a preferred embodiment, the cells are infected with the parvovirus with a MOI of 0.5×10² to 5×10² PFU/cell, or rather 0.01 or 0.05 PFU/cell. In one specific embodiment of the present invention, the NB-324K cells are seeded with a cell density from 5.0×10³ to 8.0×10³ cells/cm², preferably 5.0×10³ or 8.0×10³ cells/cm², more preferably 5.0×10³ cells/cm². In a preferred embodiment, the virus stock of wild-type H-1PV is included in the fresh animal component-free cell culture medium of the 100% medium exchange. Thus, for non-simultaneous seeding and infection, the TOI is after 2 to 6 days of cell expansion, preferably at day 3 to 6 after cell expansion of the NB-324K cells has been started. In a specific embodiment, another medium exchange to 0% FBS on day 2 post infection is performed and the cells are harvested after 3 to 5 days post infection, preferably after 4 days post infection (see FIG. 1).

Comparing the simultaneous and non-simultaneous seeding and infection process, the non-simultaneous seeding and infection process is preferred according to the present invention because the seeding density is reduced from 2.0×10⁴ to 5.0×10⁴ cells/cm² to 5.0×10³ to 8.0×10³ cells/cm². By adapting to VP-SFM™ medium in the non-simultaneous seeding and infection process, the seeding density could be reduced again from 8.0×10³ to 5.0×10³ cells/cm so that non-simultaneous infection is preferred. In this way, the cell expansion is reduced and fewer resources are needed for the seed train, which lowers the production costs.

In order to avoid undesired products of animal origin, the inventors compared a two-step with a three-step FBS reduction strategy using one or two 100% medium exchanges, wherein FIG. 1 provides an overview of all medium exchange strategies: “5-5% FBS”, “2-0% FBS”, “2-1-0% FBS”, and “2-0-0% FBS” strategy.

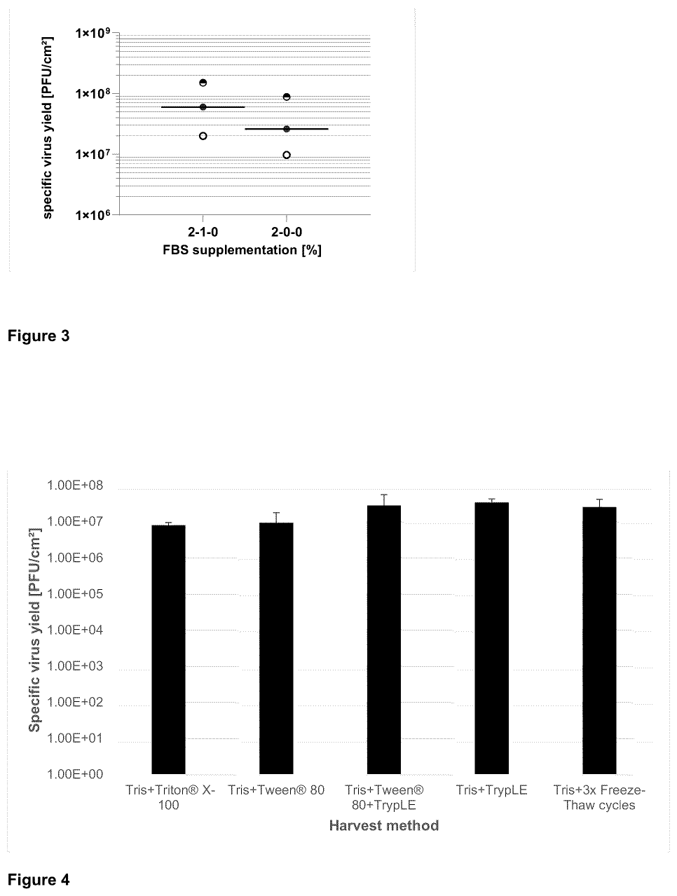

According to the present invention, all strategies preferably start with a method for cultivation or expansion of NB-324K cells, which are grown with 5% FBS in the seed train, then transferred in a culture vessel, in which animal component-free cell culture medium supplemented with 2% animal serum is laid before, for 2 to 6 days, preferably 3 to 6 days. After cell expansion for 2 to 6 days, preferably 3 to 6 days, one 100% medium exchange is performed. As already mentioned above, the time of infection (TOI) is either at seeding or after the cell expansion for 2 to 6 days, preferably 3 to 6 days, i.e. non-simultaneous with the 100% medium exchange. The 100% medium exchange is performed with new animal component-free cell culture medium supplemented with 0%, 1% or 2% FBS, preferably 0% or 1% FBS (see FIG. 3, “2-0-0% FBS” or “2-1-0% FBS” strategy), wherein the new animal component-free cell culture medium is VP-SFM™ medium. Applying the two step-process, when infection is done with e.g., 0% FBS (2-0% FBS), the inventors detected a resulting virus yield that was similar to that for 5% FBS supplementation (5-5% FBS) over the whole process (see FIG. 2). Thus, according to the present invention, the method is performed with at least one 100% medium exchange with new animal component-free cell culture medium supplemented with 0% FBS.

In the three-step strategy, on day 2 to 6, preferably on day 3 to 6 after seeding or rather after the cell expansion has been started, the one 100% medium exchange with new animal component-free cell culture medium supplemented with 0% or 1% FBS was followed by another 100% medium exchange with new animal component-free cell culture medium supplemented without FBS on day 1 to 3, preferably on day 2 postinfection (“2-1-0% FBS” or “2-0-0% FBS). Applying the three step-strategy 2-1-0% FBS, an approximately 0.3 log PFU/cm² increase could be demonstrated as compared to the 2-0-0% FBS strategy (see FIG. 3) and, thus, the 2-1-0% FBS strategy is preferred in the present invention. In summary, the depletion of FBS over the method as presently claimed is feasible with the highest virus yield achieved by applying the 2-1-0% FBS strategy.

Therefore, the method of the present invention preferably uses the optimized culture medium VP-SFM™, suitable for FBS-free production and an at least two-step medium exchange strategy in order to reduce the required amount of FBS by up to 80% for a FBS-free harvest at a comparable virus yield. With an additional medium exchange at 2 days post infection and a three-step FBS reduction from 2% to 1% and to 0%, a production yield boost of approximately 0.3 log was achieved, while still reducing the FBS needed by up to 40%. By applying this strategy during the experiments resulting in the present invention, a high virus production yield with a FBS-free harvest and fewer impurities for the downstream process could be achieved.

To simplify the large-scale H-1PV production with adherent cells, such as with the anchorage-dependent producer cell line NB-324K, the inventors employed carriers. In a preferred embodiment, microcarriers are provided for suspension cultures, wherein macrocarriers are intended for a fixed-bed bioreactor. Thus, the term “carrier(s)” in the present invention is used when both systems are discussed. The characterization of the microcarriers and macrocarriers according to the present invention is shown in Table 2, respectively.

TABLE 2
Overview Microcarriers and Macrocarriers

| | | | Abbrevi- | | |
|---|---|---|---|---|---|
| Carrier | Brand | Name | ation | Charge | Structure |
| Microcarrier | Pall | SoloHill ® Hillex ® II | HII | cationic | high-density, |
| for | | | | (DEAE Group) | solid |
| Suspension | | SoloHill ® Star-Plus | SP | neutral | solid |
| | | SoloHill ® Plastic | P | neutral | solid |
| | | SoloHill ® Plastic Plus | PP | cationic (DEAE- | solid |
| | | | | Group) | |
| | Corning | Enhanced attachment | EA | neutral | solid |
| | | CellBIND ® | | | |
| | | Low Concentration | SII | RGD* modified | sold |
| | | Synthemax ® II | | | |
| | GE | Cytodex ® 1 | CD1 | cationic (DEAE- | solid |
| | Healthcare | | | Group) | |
| | | Cytopore ™ 1 | CP1 | cationic (DEAE- | porous |
| | | | | Group) | |
| | | Cytopore ™ 2 | CP2 | cationic (DEAE- | porous |
| | | | | Group) | |
| Macrocarrier | Eppendorf | Fibra-Cel ® | FC | neutral | macroporous |
| for fixed bed | Pall | macrocarrier from | iC | neutral | macroporous |
| | | iCELLis ® | | | |

*RGD-containing sequence from the human ECM protein vitronectin, KGGPQVTRGDVFTMP, which promotes adhesion in a variety of cells

For simultaneous seeding and infection according to the present invention, the inventors provided a culture vessel in which microcarrier or macrocarrier are laid before, preferably in the animal component-free cell culture medium, which is supplemented with 2% animal serum. Then, the method of the present invention is performed as described before. Thus, very briefly, NB-324K cells, which are grown with 5% FBS in the seed train, are added in a culture vessel, in which animal component-free cell culture medium, which is supplemented with 2% animal serum and microcarriers or macrocarriers are laid before, wherein a virus stock of wild-type H-1PV is used to infect the NB-324K cells, preferably with a MOI of 0.5×10² to 5×10² PFU/cell, or rather 0.01 or 0.05 PFU/cell. According to the present invention, the NB-324K cells are seeded and infected at a cell density from 2.0×10⁴ to 5.0×10⁴ cells/cm², preferably 4.0×10⁴ cells/cm² with the parvovirus, wherein the cells are harvested after 3 to 5 days post infection, preferably after 4 days post infection.

For simultaneous seeding and infection, the inventors propose the addition of carrier between about 8 to 12 cm², preferably about 9.5 to 11.5 cm², more preferably 10 cm² or 11.3 cm², which is added per well

For non-simultaneous seeding and infection, the inventors provided a culture vessel in which a microcarrier or a macrocarrier are laid before, preferably in the animal component-free cell culture medium, which is supplemented with 2% animal serum. Then, the method of the present invention is performed as described before. Thus, very briefly, NB-324K cells, which are grown with 5% FBS in the seed train, are added in a culture vessel, in which animal component-free cell culture medium, which is supplemented with 2% animal serum and microcarriers or macrocarriers is laid before. According to the present invention, the inventors let the cells grow then for 2 to 6 days, preferably 3 to 6 days (“cell expansion”). After 2 to 6 days, preferably 3 to 6 days of cell expansion, the animal component-free cell culture medium supplemented with 2% animal serum is completely exchanged (“100% medium exchange”) with new animal component-free cell culture medium supplemented with 1% FBS and fresh microcarrier, preferably such that the total growth area is doubled. In another preferred embodiment, the virus stock of wild-type H-1PV is also included in the new animal component-free cell culture medium of the 100% medium exchange. Thus, for non-simultaneous seeding and infection, the time of infection (TOI) is after 2 to 6 days of cell expansion, preferably at day 3 to 6 after cell expansion of the NB-324K cells has been started. The medium exchange to 0% FBS takes place on day 1 to 3 after cell infection, preferably on day 2 after cell infection. In a specific embodiment, the cells are harvested after 3 to 5 days after TOI, i.e. post infection, preferably after 4 days post infection (see FIG. 1).

For non-simultaneous seeding and infection, the inventors propose between about 4 to 6 cm², preferably 5 cm² of carrier, which is added per well.

As already stated above, the characterization of the microcarriers and macrocarriers according to the present invention, respectively, is shown in Table 2. With the exception of porous Cytopore™ 1 and Cytopore™ 2, all microcarriers showed satisfactory cell growth. Furthermore, all microcarriers showed promising bead-to-bead cell transfer capability during cell growth and virus production, where the addition of fresh microcarrier allows further cell propagation skipping trypsination step.

Thus, according to the present invention microcarriers that are not porous are preferably used because the capability of bead-to-bead transfer without trypsination suggests good cell expansion capability in scaled-up seed trains.

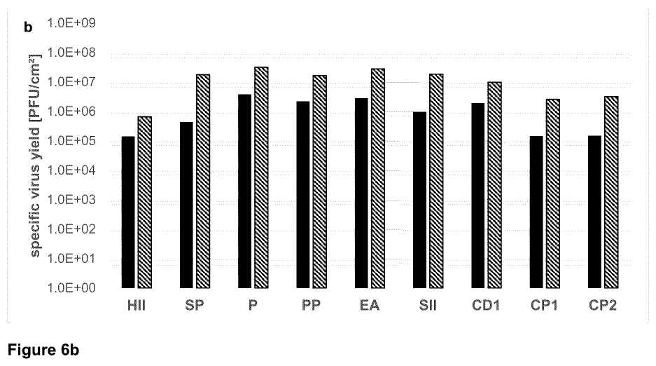

A wide range of parameters such as seeding and process agitation, carrier densities, cell-seeding densities, MOI, TOI, with/without bead-to-bead transfer, cell culture volume per vessel, and a medium exchange regimen were tested in order to find the preferred carriers (Tables 3 and 4). The results of virus production on carriers for preferred carrier selection are summarized in FIGS. 6a+b.

TABLE 3
Summary of tested parameters concerning carrier

| Parameter | Parameter range |
|---|---|
| Seeding RPMs | 0-100 |
| Process RPMs | 0-100 |
| Carrier density [cm²/ml] | 5-20 |
| Seeding density [cells/cm²] | 5E3-4E4 |
| MOI | 0.01, 0.05 |
| TOI [day] | 0, 3, 6, 9, 13, 16 |
| Bead-to-bead transfer | (+), (−) |
| Medium exchange at TOI | 50%, 100% |
| Fed batch | (+), (−) |

TABLE 4
Summary of tested medium volumes with
different vessels in carrier experiments

| | | | Medium |
|---|---|---|---|
| System | Scale | | volume [ml] |
| Plate | 24-well | | 1 |
| Erlenmeyer | 125 | ml | 17.5-40 |
| Flask | 1 | L | 150-300 |
| | 5 | L | 1500 |
| Spinner | 250 | ml | 100 |
| flask | 500 | ml | 100 |

Thus, in a preferred embodiment of the present invention, the microcarrier is a cross-linked dextran matrix with positively charged DEAE (N,N-diethylaminoethyl)-groups distributed throughout the matrix (Cytodex® 1; CD1). In another preferred embodiment of the present invention, the microcarrier represents the “Enhanced attachment CellBIND® (EA, enhanced attachment surface treatment infuses the surface of the microcarriers with oxygen). In an alternatively preferred embodiment, the macrocarrier is a polypropylene and polyester nonwoven fiber (Fibra-Cel®; FC) or a nonwoven, hydrophilized polyethylene terephthalate (PET) macrocarrier from iCELLis® (iC; or iC-500 m²).

For simultaneous and nonsimultaneous seeding and infection, the microcarrier CD1 and the macrocarrier iC represent the preferred carriers in one embodiment of the present invention. Furthermore, the macrocarriers FC and iC represent alternatively preferred embodiments of the present invention because they show a good production yield in suspension cultures and fixed-bed bioreactors, wherein in fixed-bed bioreactors a higher production yield is achieved.

Applying the method of the present invention, the majority of infective virus particles are cell-associated at the time of harvest. To harvest H-1PV, a freeze-thaw cell lysis in Tris-EDTA buffer (VTE) (Leuchs 2016) or Tris-HCl buffer (VT) (Leuchs 2017) was previously reported for stationary cultures. The freeze-and-thaw method, however, is limited for large-scale production with adherent cells on carriers such that a scalable cell lysis is required. Therefore, in the experiments resulting in the present invention, an alternative cell lysis method has been developed. In an embodiment, the present invention refers to the cell lysis with lysis buffer containing Tris, MgCl₂ and recombinant cell-dissociation enzyme TrypLE™ with Tween® 80, which is eco-friendlier than Triton® X-100 and which achieves a similar or higher production yield. However, Tween® 80 may be omitted from the lysis buffer recipe without negative influence on the virus yield.

According to the present invention, the lysis of the NB-324K cells in step (g) is performed 3 to 5 days post-infection with “lysis buffer” containing 1-100 mM Tris, 1-10 mM MgCl₂, 2.5-10% TrypLE™, pH 9-10 with or without 0.1-1% Tween® 80, preferably with lysis buffer containing 25 mM Tris, 5 mM MgCl₂, 5% TrypLE™ pH 10 for 1 h at 40° C. without CO₂.

According to the present invention, the lysis with lysis buffer in step (g) is followed by wash with “wash buffer” containing 1-100 mM Tris, 1-10 mM MgCl₂, pH 9-10, preferably with wash buffer containing 25 mM Tris, 5 mM MgCl₂ pH 10, wherein the lysis and wash buffer are pooled together and, wherein the lysis and wash step, i.e. step (g) of the present invention results in buffer containing 1-100 mM Tris, preferably 25 mM Tris; 1-10 mM MgCl₂, preferably 5 mM MgCl₂; 2.5-10% TrypLE™, preferably 2.5% TrypLE™, pH 10, with or without 0.1-1% Tween®80, preferably less than 0.25% Tween® 80.

“TrypLE™” is a reagent having highly purified, recombinant cell-dissociation enzymes that replace porcine trypsin (GIBCO, USA).

Taken together, medium optimization and the three-step medium exchange strategy and harvest with lysis buffer containing Tris, MgCl₂ and recombinant cell-dissociation enzyme TrypLE™ with or without Tween® 80, preferably without Tween® 80, constitute a solid basis for upscaling production.

In summary, with the optimized cell culture medium VP-SFM™ and the new medium exchange strategy, the inventors established a reduction in seeded cell density and FBS, leading to a FBS-free harvest, wherein the tested carriers are best suited for a high H-1PV yield, cell growth, and bead-to-bead transfer capability. They also demonstrated feasible, carrier-based production and successfully scaled up the process from 24-well plates to Erlenmeyer, Spinner flasks and iCellis nano.

The following examples are intended to illustrate, but not to limit the invention. While such examples are typical of those that might be used, other methods known to those skilled in the art may alternatively be utilized.

### Example 1

### Material and Methods

### Cell Line and Cell Culture Media

NB-324K human newborn kidney cells (Tattersall 1983) transformed with simian virus 40 were cultured at 37° C. in VP-SFM™ medium (Thermofisher, USA) with 5% FBS in a 5% CO₂ atmosphere. Cell culture media were supplemented with 100 U/ml penicillin, 100 μg/ml streptomycin, and 4 mM L-glutamine (see Table 1).

### Virus Stock, Time of Infection

A virus stock of wild-type H-1PV, produced and purified in house, was used to infect the cells with a MOI of 0.01 or 0.05 plaque-forming units (PFU) per cell. Time of infection (TOI) was either at seeding, based on Countess cell count (simultaneous infection and seeding) or at day 3 to 6 (non-simultaneous infection).

### Cell Cultivation and Virus Production in Stationary Culture

Simultaneous seeding and infection: 3.6E4 cells/cm² were seeded in T175 flask with VP-SFM™ full, VP-SFM™ w/o FBS (see table 1) and simultaneously infected. Cells were harvested at 4 days post infection (dpi).

Non-simultaneous seeding and infection: 7.9E3 cells/cm² were seeded in T175 flask with VP-SFM™ full, or VP-SFM™ cell expansion medium (see Table 1). After 3 days of cell expansion, the medium was completely exchanged with VP-SFM™ w/o FBS or VP-SFM™ infection medium. Simultaneously, cells were infected with a MOI of 0.01 or 0.05 according to the Countess cell count of a reference T175 flask. On day 2 postinfection, another 100% medium exchange with VP-SFM™ w/o FBS was performed for cells that had been in VP-SFM™ infection medium since day 3. Cells were harvested on day 4 postinfection by freeze-thaw.

### Carrier Preparation

Microcarriers and macrocarriers (termed “carriers” when both systems are discussed) characterization is shown in table 2. Microcarriers were handled and stored in bottles that were siliconized with Sigmacote (Sigma-Aldrich, Germany) according to the manufacturer's instructions. Noncationic microcarriers were hydrated and autoclaved in aqua ad. injectable (B. Braun, Germany), cationic microcarriers in 1×PBS without Ca²⁺ and Mg²⁺, according to the manufacturer's instructions. Macrocarriers were sterile when supplied and hydrated in cell culture medium for 30 min at 37° C. before use.

### Cell Cultivation and Production Systems for Carriers

Screening in 24-well plates: Screening experiments of the carriers were performed in 24-well, ultra-low attachment plates (Corning, Germany) with 1 ml VP-SFM™ full per well, at 37° C., 5% CO₂, and 100 rpm orbital agitation with Max Q 2000 CO₂ Plus (ThermoFisher Scientific, USA). Stationary controls were treated like carrier samples but seeded in 6-well plates (9.6 cm² growth area) with 2 ml cell culture medium per well without agitation.

For simultaneous seeding and infection, 10 cm² or 11.3 cm² (macrocarrier from iCELLis® were cut in half) of carrier were added per well to three wells. Then, 1 ml VP-SFM™ full with NB-324K cells, corresponding to a seeding density of 4E4 cells/cm², was added to each well and infected with MOI of 0.01. Cells were harvested 4 days postinfection with Triton® X-100.

For nonsimultaneous seeding and infection, 5 cm² of growth area was added per well in 2 wells. Then, 1 ml VP-SFM™ full with NB-324K cells, corresponding to a seeding density of 2E4 or 4E4 cells/cm², were added to each well. On day 3, both wells per carrier were pooled and the nuclei from a sample counted. Cells were infected (MOI of 0.01) by adding fresh cell culture medium VP-SFM™ full, including the virus and fresh carrier, doubling the total growth area from 10 cm² to 20 cm² and the cell culture volume from 2 ml to 4 ml for each pool. The pool of spent and fresh carrier was then split into 3 wells with a 5 cm² growth area and 1 ml cell culture medium per well. Cells were harvested 4 days postinfection on day 7 with Triton® X-100.

Microcarriers in Erlenmeyer flask: After screening, Enhanced attachment (EA) and Cytodex® 1 (CD1) microcarriers were selected for upscaling experiments in a 125-ml Erlenmeyer flask with 40 ml VP-SFM™ full and 10 cm²/ml growth area, at 37° C., 5% CO₂, and 60-70 rpm orbital agitation with Max Q 2000 CO₂ Plus (ThermoFisher Scientific, USA). Here, 2E4 cells/cm² were seeded and agitation was reduced to 0 rpm for 30 min or to 30 rpm for 3 h to promote cell attachment. On day 3, a sample was taken to determine cell density with nuclei count for virus infection (MOI of 0.01) and virus was added during a 50% medium exchange on the same day with fresh VP-SFM™ full.

Virus Production in Erlenmeyer Flasks with Macrocarriers

Fibra-Cel® and macrocarrier from iCELLis© were also tested in 125-ml Erlenmeyer flasks with parameters similar to those described for the microcarriers. However, orbital agitation was 30-100 rpm and agitation during seeding was either 100 rpm or a cycle of 40 rpm for 1 minute and then 0 rpm for 30 min, which was repeated four times to a total seeding time of 2 h.

Virus Production in Spinner Flasks with Carriers

EA and CD1 microcarriers were further scaled up in a 250-ml Spinner flask (Integra Biosciences, Switzerland) and Fibra-Cel® and macrocarrier from iCELLis® in a 500-ml Spinner flask (Integra Biosciences, Switzerland) with 100 ml VP-SFM™ full and 10 cm²/ml growth area, at 37° C., 5% CO₂, and 15-30 rpm agitation. Then, 2E4 cells/cm² were seeded and agitation was reduced to 0 rpm for 30 min or a cycle of 40 rpm for 1 minute and then 0 rpm for 30 min, which was repeated four times to a total seeding time of 2 h. On day 3, a sample was taken to determine cell density with nuclei count for virus infection (MOI of 0.01 or 0.05) and virus was added during a 50% medium exchange with VP-SFM™ full on the same day. Cells were harvested 4 days postinfection.

Virus Production in iCELLis® Nano

The iCELLis® nano system was tested in 0.53 m² and 4 m² fixed-bed sizes. After preparing the fixed bed according to the manufacturer's instructions, bioreactors were filled with 850 ml VP-SFM™ Cell expansion medium supplemented with 2% FBS. For the 4 m² fixed bed, a recirculation loop supplying an additional 3150 ml VP-SFM™ cell expansion medium was connected. Then, 5E3 cells/cm² were seeded for the 0.53 m² fixed bed or 9E3 cells/cm² for the 4 m² fixed bed and maintained at 37° C., pH 7.3, above 30-40% dissolved oxygen. After 3-6 days of cell expansion, several macrocarriers were taken from the top of the fixed bed and cells were counted, followed by infection with an MOI of 0.01 during a 100% medium exchange to VP-SFM™ infection medium supplemented with 1% FBS. Two days postinfection, an additional 100% medium exchange to VP-SFM™ without FBS medium was performed. Cells were harvested 4 days postinfection.

### Harvest

For carrier cultures plated in wells, Erlenmeyer or Spinner flasks, the cell culture medium was removed 4 days postinfection and then treated for 30 min at 37° C. with 0.02 ml/cm² 1% Triton® X-100, 0.1 M Tris, pH 9.5, for cell lysis.

For iCELLis® nano cultures, the cell culture medium was removed 4 days post infection. Then, the cells in the fixed bed can be rinsed with PBS and lysed with 0.094 ml/cm² (0.53 m²) or 0.014 ml/cm² (4 m²) lysis buffer 25 mM Tris, 5 mM MgCl₂, 5% TrypLE™ pH 10 with or without 0.5% Tween® 80, for 1 h at 40° C. w/o CO₂ and rinsed with wash buffer 25 mM Tris, 5 mM MgCl₂ pH 10. Lysis and wash are pooled together resulting in 25 mM Tris, 5 mM MgCl₂, 2.5% TrypLE™ pH 10 with or without 0.25% Tween® 80, preferably without Tween® 80.

For stationary cultures, cell lysis via a freeze/thaw process was performed. The medium was removed and infected cells were washed with 1×PBS. The medium supernatant and detached cells were centrifuged for 5 min at 5000× g. The pellet was washed with PBS, re-suspended with 0.02 ml/cm² 0.05 M Tris-HCl, pH 8.7 (VT), for 30 min at 37° C., and subjected to three freeze (liquid nitrogen) and thaw (37° C.) cycles. In addition, the following steps were taken during medium optimization and FBS reduction: After centrifugation for 5 min at 5000× g, cell debris was discarded. The cell lysate was then sonicated at 48 W for 1 min with a Sonorex Super 10 P ultrasonic homogenizer (Bandelin, Germany) and treated with DNAse (50 U/ml, Sigma, Germany) after adding 5 mM MgCl₂ for 30 min at 37° C. to eliminate nonencapsidated viral DNA and contaminating host cell DNA.

### Virus Yield Quantification

Virus was quantified by performing a plaque formation assay for infectious particles (see Leuchs (2016), for a description of the method).

### Example 2

### Comparison of a Two-Step with a Three-Step FBS Reduction Strategy Using 100% Medium Exchanges

Afterwards, the inventors compared a two-step with a three-step FBS reduction strategy using 100% medium exchanges. See FIG. 1 for an overview of all medium exchange strategies with VP-SFM™ medium. All strategies started with 5% FBS in the seed train, followed by three days of cell expansion in 5% or 2% FBS and a 100% medium exchange with simultaneous infection on day 3 after seeding. In the two step-process, when infection is done with 0% FBS (2-0%) the resulting virus yield of 1.0E7 PFU/cm² was similar to that for 5% FBS supplementation (5-5%) over the whole process (see FIG. 2). In the three-step strategies, the 100% medium exchange on day 3 after seeding with 2% FBS was supplemented with either 1% FBS (2-1-0%) or without FBS (2-0-0%), followed by a second 100% medium exchange without FBS on day 5 for both. With three independent experiments, it has been demonstrated that the 2-1-0% FBS strategy resulted in approximately 0.3 log PFU/cm² increase in yield up to 7.7E7 PFU/cm², as compared to the 2-0-0% FBS strategy (FIG. 3). In summary, the results indicate that depletion of FBS over the process is feasible with the highest virus yield achieved by applying the 2-1-0% FBS strategy. However, FBS includes components needed for a high virus yield which cannot be supplied by VP-SFM™ alone.

### Example 3

### Chemical Cell Lysis Needed for Large Scale H-1PV Harvest

In the method according to the present invention, the majority of infective virus particles are cell-associated at the time of harvest. To harvest H-1PV, a freeze-thaw cell lysis in Tris-EDTA buffer (VTE) (Leuchs 2016) or Tris-HCl buffer (VT) (Leuchs 2017) was previously reported for stationary cultures. For large-scale production with adherent cells on carriers, a scalable cell lysis is required. Therefore, an alternative cell lysis method with lysis buffer containing Tris, MgCl₂ and recombinant cell-dissociation enzyme TrypLE™ with or without Tween® 80 resulting in a satisfactory virus yield >2.0E7 PFU/cm² (see FIG. 4) has been developed.

Taken together, medium optimization and the three-step medium exchange strategy with Triton® X-100 lysis or lysis buffer containing Tris, MgCl₂ and recombinant cell-dissociation enzymes with or without Tween® 80 constitute a solid basis for upscaling production.

### Example 4

### Screening of Cell Growth on Microcarriers and Macrocarriers in 24-Well Scale are Capable of Bead-to-Bead Cell Transfer

For large-scale production with adherent cells, microcarriers can be employed for suspension culture or macrocarriers for a fixed-bed bioreactor. The inventors screened cell growth, bead-to-bead-transfer capability, and virus yield for eleven carrier types. Direct cell counting on carriers was difficult. Therefore, they measured glucose consumption as an indicator of growth.

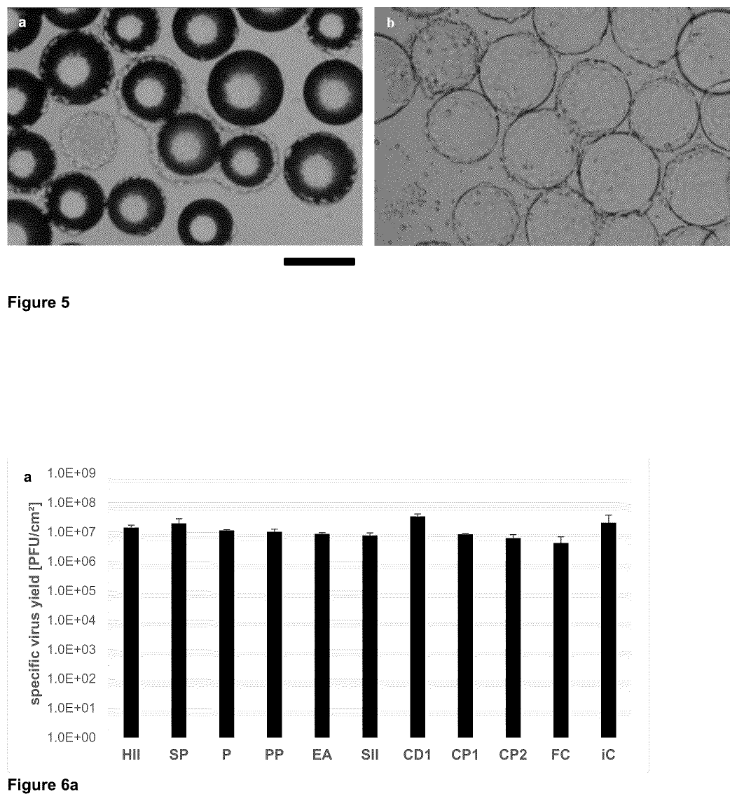

Some cell lines are capable of building individual cell bridges from a confluent microcarrier to a fresh one for continued cell growth. This bead-to-bead transfer can facilitate seed train cell expansion without trypsination because fresh microcarriers only need to be added. To test bead-to-bead transfer capability, cells were seeded on microcarriers and more microcarriers were added with the fresh cell culture medium by a 1:2 split on day 4 and day 7. Trypan blue cell count of microcarriers after trypsination was performed on day 4 before the 1:2 split and on day 10. In FIG. 5, the bead-to-bead transfer with cell bridges is shown with microcarrier EA and CD1. The capability of bead-to-bead transfer without trypsination suggests good cell expansion capability in scaled-up seed trains with NB-324K cells.

### Example 5

### Cytodex® 1 and Macrocarrier from iCELLis® Show Highest H-1PV Yield

After characterizing cell growth on the carriers, the inventors applied different production strategies (simultaneous infection/nonsimultaneous seeding and infection, with or without bead-to-bead transfer) to identify conditions most suited for high virus production of H-1PV.

Simultaneous infection and seeding of carriers in 24-well, ultra-low attachment plates resulted in similar virus yield for most microcarriers (HII, SP, P, PP, EA, SII, CP1, CP2). Highest yield was achieved with microcarrier CD1 and macrocarrier iC, 3.4E7 PFU/cm² and 2.0E7 PFU/cm², respectively (see FIG. 6a). Macrocarrier FC had the lowest yield of 4.2E6 PFU/cm².

For non-simultaneous seeding and infection, in addition to the 4E4 cells/cm² density, a 50% reduced seeding density of 2E4 cells/cm² was tested for higher yield. Three days after seeding, the cells were infected and a 1:2 split, adding fresh cell culture medium and microcarriers to maintain volume and microcarrier density, was performed for bead-to-bead transfer. Virus yield was highest at a seeding density of 4E4 cells/cm², which was similar for most microcarriers compared to simultaneous seeding and infection without bead-to-bead transfer, with the exception of the solid HII and the porous CP1 and CP2 carriers (see FIG. 6b). Taken together, high virus production seems possible with all tested strategies but requires a seeding density of 4E4 cells/cm². In addition, HII and porous microcarriers CP1 and CP2 only showed a high yield for simultaneous infection and seeding without bead-to-bead transfer.

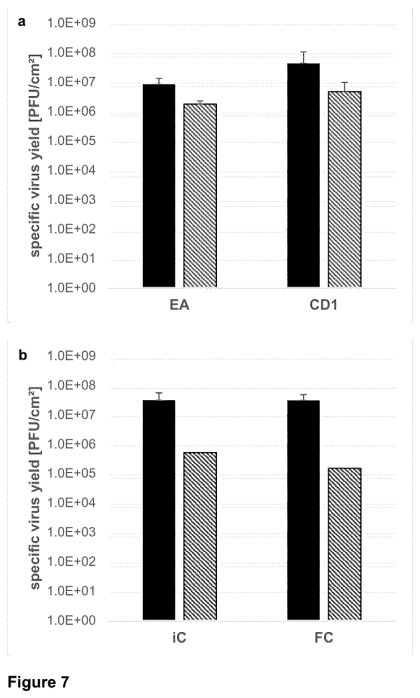

Upscaling from 24-well plate format up to 100 ml with Erlenmeyer and Spinner flasks was examined with two selected microcarriers and two macrocarriers. The microcarrier CD1 showed the highest yield, while microcarrier EA surface is comparable to that for 10-layer CellSTACK® chambers. Both macrocarriers for fixed-bed bioreactors were also chosen for upscaling experiments, due to possible limitations associated with 24-well plate, small-scale testing. A wide range of parameters such as seeding and process agitation, carrier densities, cell-seeding densities, MOI, TOI, with/without bead-to-bead transfer, cell culture volume per vessel, and a medium exchange regimen were tested (see Table 3 and 4). However, only the most promising parameters with 40 ml cell culture medium in an Erlenmeyer flask and 100 ml in a Spinner flask are shown (see FIGS. 7a and b).

The microcarrier CD1 reached a yield level of 4.3E7 PFU/cm² in the Erlenmeyer flask, but it was 1 log less when upscaled in the Spinner flask. The microcarrier EA had a lower virus yield than CD1 in all systems.

A 3.0E7 PFU/cm² virus yield was achieved with macrocarrier iC and FC in the Erlenmeyer flask, a macrocarrier density of 10 cm²/ml, and a total cell surface of 400 cm². However, when upscaled to the Spinner flask, yield was below 1.0E6 PFU/cm² with 10 cm²/ml and 1000 cm² cell surface without bead-to-bead transfer. The results of CD1 and both macrocarriers in the Erlenmeyer flask confirm that high virus yield is possible in suspension and these carriers are the best candidates for further upscaling.

### Example 6

### First Production in iCELLis® Nano

Assuming a yield of 3.0E7 PFU/cm² that was generated in the Erlenmeyer flask, with the iCELLis® 500 m² fixed bed, a batch yield of 1.5E14 PFU can be expected (corresponding to 15,000 doses, each with 1E10 PFU). Therefore, virus production was tested in the downscaled iCELLis® nano system.

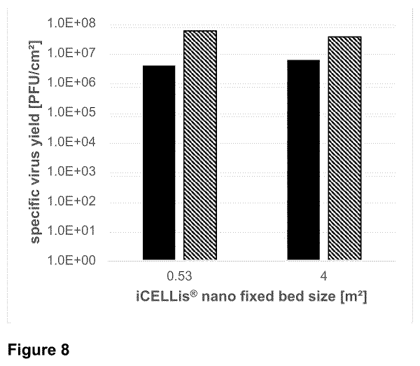

For production in iCELLis® nano benchtop bioreactor, the newly developed medium exchange strategy 2-1-0% FBS was used. Here, 0.53 m² and 4 m² fixed-bed sizes were tested and resulted in 3.7E6 PFU/cm² and 5.7E6 PFU/cm², respectively (see FIG. 8). Both fixed beds had a comparable, but approximately one log step lower yield than did macrocarrier from iCELLis® in the Erlenmeyer flask and stationary run control.

### LIST OF REFERENCES

ADDIN EN.REFLIST Angelova A L, Aprahamian M, Balboni G, Delecluse H J, Feederle R, Kiprianova I, Grekova S P, Galabov A S, Witzens-Harig M, Ho A D, Rommelaere J, Raykov Z (2009a) Oncolytic rat parvovirus h-lpv, a candidate for the treatment of human lymphoma: In vitro and in vivo studies. Mol Ther 17(7):1164-72 doi:10.1038/mt.2009.78
Angelova A L, Aprahamian M, Grekova S P, Hajri A, Leuchs B, Giese N A, Dinsart C, Herrmann A, Balboni G, Rommelaere J, Raykov Z (2009b) Improvement of gemcitabine-based therapy of pancreatic carcinoma by means of oncolytic parvovirus h-lpv. Clin Cancer Res 15(2):511-9 doi:10.1158/1078-0432.CCR-08-1088
Cotmore S F, Agbandje-McKenna M, Chiorini J A, Mukha D V, Pintel D J, Qiu J, Soderlund-Venermo M, Tattersall P, Tijssen P, Gatherer D, Davison A J (2014) The family parvoviridae. Arch Virol 159(5):1239-47 doi:10.1007/s00705-013-1914-1
Dupressoir T, Vanacker J M, Cornelis J J, Duponchel N, Rommelaere J (1989) Inhibition by parvovirus h-1 of the formation of tumors in nude mice and colonies in vitro by transformed human mammary epithelial cells. Cancer Res 49(12):3203-8
Faisst S, Guittard D, Benner A, Cesbron J Y, Schlehofer J R, Rommelaere J, Dupressoir T (1998) Dose-dependent regression of hela cell-derived tumours in scid mice after parvovirus h-1 infection. Int J Cancer 75(4):584-9 doi:10.1002/(sici)1097-0215(19980209)75:4<584::aid-ijc15>3.0.co;2-9
Fukuhara H, Ino Y, Todo T (2016) Oncolytic virus therapy: A new era of cancer treatment at dawn. Cancer Sci 107(10):1373-1379 doi:10.1111/cas.13027
Geletneky K, Hajda J, Angelova A L, Leuchs B, Capper D, Bartsch A J, Neumann J O, Schoning T, Husing J, Beelte B, Kiprianova I, Roscher M, Bhat R, von Deimling A, Bruck W, Just A, Frehtman V, Lobhard S, Terletskaia-Ladwig E, Fry J, Jochims K, Daniel V, Krebs O, Dahm M, Huber B, Unterberg A, Rommelaere J (2017) Oncolytic h-1 parvovirus shows safety and signs of immunogenic activity in a first phase i/iia glioblastoma trial. Mol Ther 25(12):2620-2634 doi:10.1016/j.ymthe.2017.08.016
Geletneky K, Huesing J, Rommelaere J, Schlehofer J R, Leuchs B, Dahm M, Krebs O, von Knebel Doeberitz M, Huber B, Hajda J (2012) Phase i/iia study of intratumoral/intracerebral or intravenous/intracerebral administration of parvovirus h-1 (parvoryx) in patients with progressive primary or recurrent glioblastoma multiforme: Parvoryx0l protocol. BMC Cancer 12:99 doi:10.1186/1471-2407-12-99
Geletneky K, Kiprianova I, Ayache A, Koch R, Herrero Y C M, Deleu L, Sommer C, Thomas N, Rommelaere J, Schlehofer J R (2010) Regression of advanced rat and human gliomas by local or systemic treatment with oncolytic parvovirus h-1 in rat models. Neuro Oncol 12(8):804-14 doi:10.1093/neuonc/noq023
Hajda J, Leuchs B, Angelova A L, Frehtman V, Rommelaere J, Mertens M, Pilz M, Kieser M, Krebs O, Dahm M, Huber B, Engeland C E, Mavratzas A, Hohmann N, Schreiber J, Jager D, Halama N, Sedlaczek O, Gaida M M, Daniel V, Springfeld C, Ungerechts G (2021) Phase 2 trial of oncolytic h-1 parvovirus therapy shows safety and signs of immune system activation in patients with metastatic pancreatic ductal adenocarcinoma. Clin Cancer Res doi:10.1158/1078-0432.CCR-21-1020
Leuchs B, Frehtman V, Riese M, Muller M, Rommelaere J (2017) A novel scalable, robust downstream process for oncolytic rat parvovirus: Isoelectric point-based elimination of empty particles. Appl Microbiol Biotechnol 101(8):3143-3152 doi:10.1007/s00253-016-8071-x
Leuchs B, Roscher M, Muller M, Kurschner K, Rommelaere J (2016) Standardized large-scale h-lpv production process with efficient quality and quantity monitoring. J Virol Methods 229:48-59 doi:10.1016/j.jviromet.2015.11.022
Liu S, Ruban L, Wang Y, Zhou Y, Nesbeth D N (2017) Establishing elements of a synthetic biology platform for vaccinia virus production: Biobrick design, serum-free virus production and microcarrier-based cultivation of cv-1 cells. Heliyon 3(2):e00238 doi:10.1016/j.heliyon.2017.e00238
Martinez J A A, Milian J V F, Ueti M W, Rojas-Martinez C (2020) Innovative alternatives for continuous in vitro culture of babesia bigemina in medium free of components of animal origin. Pathogens 9(5) doi:ARTN 343 10.3390/pathogens9050343
Nuesch J P, Lacroix J, Marchini A, Rommelaere J (2012) Molecular pathways: Rodent parvoviruses—mechanisms of oncolysis and prospects for clinical cancer treatment. Clin Cancer Res 18(13):3516-23 doi:10.1158/1078-0432.CCR-11-2325
Rommelaere J, Geletneky K, Angelova A L, Daeffler L, Dinsart C, Kiprianova I,
Schlehofer J R, Raykov Z (2010) Oncolytic parvoviruses as cancer therapeutics. Cytokine Growth Factor Rev 21(2-3):185-95 doi:10.1016/j.cytogfr.2010.02.011
Rourou S, van der Ark A, van der Velden T, Kallel H (2007) A microcarrier cell culture process for propagating rabies virus in vero cells grown in a stirred bioreactor under fully animal component free conditions. Vaccine 25(19):3879-3889 doi:10.1016/j.vaccine.2007.01.086 Stewart B, Wild C (2014) World cancer report 2014. International Agency for Research on Cancer
Tattersall P, Bratton J (1983) Reciprocal productive and restrictive virus-cell interactions of immunosuppressive and prototype strains of minute virus of mice. J Virol 46(3):944-55
Tree J A, Richardson C, Fooks A R, Clegg J C, Looby D (2001) Comparison of large-scale mammalian cell culture systems with egg culture for the production of influenza virus a vaccine strains. Vaccine 19(25-26):3444-50 doi:10.1016/s0264-410x(01)00053-6
van der Valk J, Bieback K, Buta C, Cochrane B, Dirks W G, Fu J, Hickman J J, Hohensee C, Kolar R, Liebsch M, Pistollato F, Schulz M, Thieme D, Weber T, Wiest J, Winkler S, Gstraunthaler G (2018) Fetal bovine serum (fbs): Past-present-future. ALTEX 35(1):99-118 doi:10.14573/altex.1705101
Wu S C, Huang G Y L, Liu J H (2002) Production of retrovirus and adenovirus vectors for gene therapy: A comparative study using microcarrier and stationary cell culture. Biotechnol Prog 18(3):617-622 doi:10.1021/bp020026p

1. A method for producing parvovirus H-1 (H-1PV), said method comprising:
(a) providing the producer cell line NB-324K;
(b) producing the seed train by growing the NB-324K cells under suitable conditions;
(c) providing a culture vessel containing animal component-free cell culture medium supplemented with 2% animal serum and microcarrier or macrocarrier at the seeding time point;
(d) seeding the NB-324K cells of step (b) in the culture vessel of step (c);
(e) performing cell expansion of the NB-324K cells;
(f) performing 100% medium exchange at the infection time point with animal component-free cell culture medium including microcarrier or macrocarrier, wherein the animal component-free cell culture medium is supplemented with 1% animal serum or without animal serum;
(g) harvesting the NB-324K cells 3 to 8 days post-infection with lysis buffer containing 1-100 mM Tris, 1-10 mM MgCl₂, 2.5-10% TrypLE™, pH 9-10 with or without 0.1-1% Tween® 80, and agitation of the lysis buffer, followed by wash with wash buffer containing 1-100 mM Tris, 1-10 mM MgCl₂, pH 9-10, wherein the lysis and wash buffer are pooled together;
(h) clarifying the parvovirus harvest by filtration;
(i) eliminating non-encapsidated viral DNA and contaminating host cell DNA by DNAse treatment;
(j) performing tangential flow filtration for buffer exchange and concentration;
(k) performing anion exchange chromatography to eliminate empty particles and most impurities;
(l) performing tangential flow filtration for buffer exchange to 0-3% Visipaque and 97-100% Ringer solution and concentration;
(m) obtaining a final formulation in 48% Visipaque/Ringer solution.

2. The method according to claim 1, wherein in step (g) the lysis buffer contains 25 mM Tris, 5 mM MgCl₂, 5% TrypLE™ pH 10 for 1 h at 40° C. without CO₂.

3. The method according to claim 1, wherein in step (g) the step of washing is performed with buffer containing 25 mM Tris, 5 mM MgCl₂ pH 10 and, wherein step (g) results in buffer containing 25 mM Tris, 5 mM MgCl₂, 2.5% TrypLE™, with or without 0.25% Tween® 80.

4. The method according to claim 1, wherein the microcarrier is a cross-linked dextran matrix with positively charged DEAE (N,N-diethylaminoethyl)-groups distributed throughout the matrix, such as Cytodex® 1 or enhanced attachment CellBIND® (EA).

5. The method according to claim 1, wherein the macrocarrier is a polypropylene and polyester non-woven fiber (Fibra-Cel®) or a nonwoven, hydrophilized polyethylene terephthalate (PET) macrocarrier (iCELLis®).

6. The method according to claim 5, wherein the NB-324K cells are seeded at a seeding cell density from 2.0×10⁴ to 5.0×10⁴ cells/cm² when seeding and infection occurs at step (d) or seeding cell density from 5.0×10³ to 8.0×10³ cells/cm² when infection occurs after 2 to 6 days of cell expansion of step (e).

7. The method according to claim 1, wherein the animal serum is heat-inactivated fetal bovine serum (FBS).

8. The method according to claim 1, wherein a second 100% medium exchange supplemented without animal serum is performed after step (f).

9. The method according to claim 8, wherein the medium of the 100% medium exchange in step (f) is supplemented with 1% FBS or without FBS and, wherein the medium of the second 100% medium exchange is supplemented without FBS.

10. The method according to claim 8, wherein the second 100% medium exchange is performed on day 1-3 post infection.

11. The method according to claim 1, wherein the animal component-free cell culture medium virus-production-serum free medium (VP-SFM™) comprising 16-22 mM glucose, 3-5 mM glutamine, 0.1-0.6 mM glutamate, 0.5-1.0 mM lactate, less than 0.3 mM ammonium and 3-10 μg/μI proteins.

12. The method according to claim 11, wherein the animal-component free cell culture medium is VP-SFM™ and is supplemented with 4 mM L-glutamine.

13. The method according to claim 1, wherein the method starts with 5% FBS in the seed train.

14. The method according to claim 1, wherein the cell lysis is performed with buffer containing Tris, MgCl₂ and recombinant cell-dissociation enzyme TrypLE™ with or without Tween® 80, pH 10.

15. The method according to claim 1, wherein the method is used for a suspension culture or a fixed-bed bioreactor.

16. The method according to claim 9, wherein the second 100% medium exchange is performed on day 1-3 post infection.
